(19) United States
(12) Plant Patent Application Publication
(10) Pub. No.: US 2017/0127590 P1
(43) Pub. Date: May 4, 2017

(54) Hop plant named 'HBC 522'

(50) ;

(71) Applicants: Eugene G. Probasco, Yakima, WA (US); Jason Perrault, Toppenish, WA (US)

(72) Inventors: Eugene G. Probasco, Yakima, WA (US); Jason Perrault, Toppenish, WA (US)

(21) Appl. No.: 15/330,749

(22) Filed: Nov. 1, 2016

Related U.S. Application Data
(60) Provisional application No. 62/285,569, filed on Nov. 2, 2015.

(51) Int. Cl. A01H 5/02

(52) U.S. Cl. PLT/236

(57) ABSTRACT

A new hop plant named ‘HBC 522’ is disclosed. ‘HBC 522’ is used for its exceptional and unique aromatic qualities.

### CROSS-REFERENCE TO RELATED APPLICATIONS

This application claims priority of co-pending provisional application Ser. No. 62/285,569 as filed on Nov. 2, 2015.

Statement regarding federally sponsored research or development: None.

Genus and species: Humulus lupulus.

Variety denomination: ‘HBC 522’.

### BACKGROUND OF THE INVENTION

‘HBC 522’ is a product of a controlled breeding program carried out by the inventors in the Yakima Valley of Washington State. ‘HBC 522’ was one of several seedlings resulting from a controlled cross made in 2006 with female parent Columbus (U.S. Plant Pat. No. 10,956) and male parent 986-2 (unpatented). A single plant of ‘HBC 522’ was selected in 2009, and in 2010 was expanded to 30 plants, which were planted in the area of Toppenish, Wash. The plants were observed and evaluated for several years, and in 2014 a 1 acre test plot of ‘HBC 522’ was established in the area of Moxee, Wash. Throughout several generations of asexual propagation, ‘HBC 522’ has been observed to retain its distinctive characteristics and remain true to type.

### BRIEF DESCRIPTION OF THE PHOTOGRAPHS

FIG. 1 illustrates a mature ‘HBC 522’ hop plant grown on a trellis.

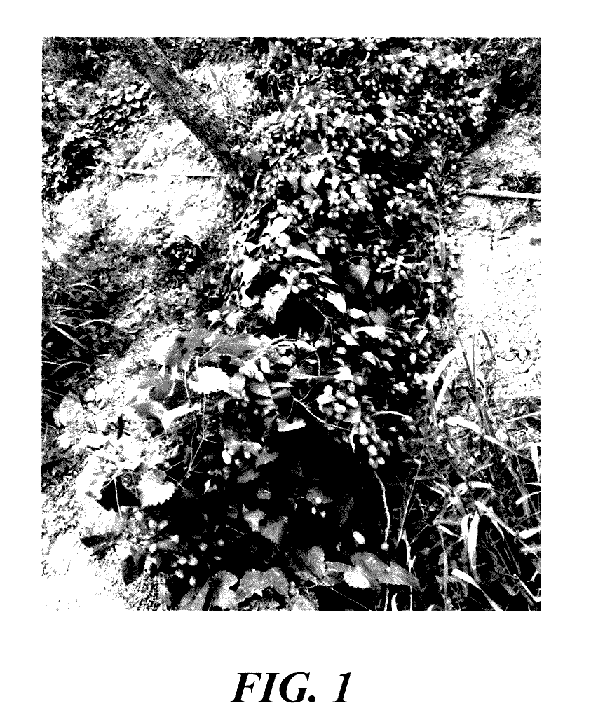

FIG. 2 illustrates whole cones and cross sections of cones of the ‘HBC 522’ hop plant.

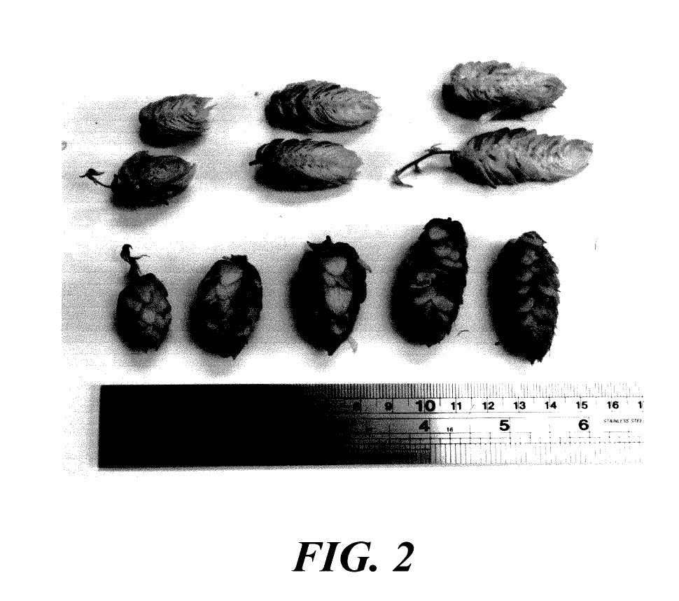

FIG. 3 illustrates the bine of a mature ‘HBC 522’ hop plant.

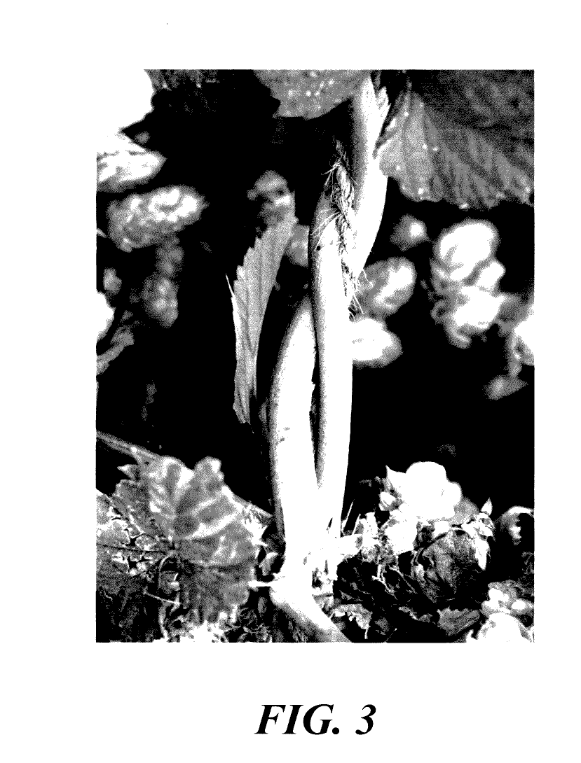

FIG. 4 illustrates cones on a mature ‘HBC 522’ hop plant.

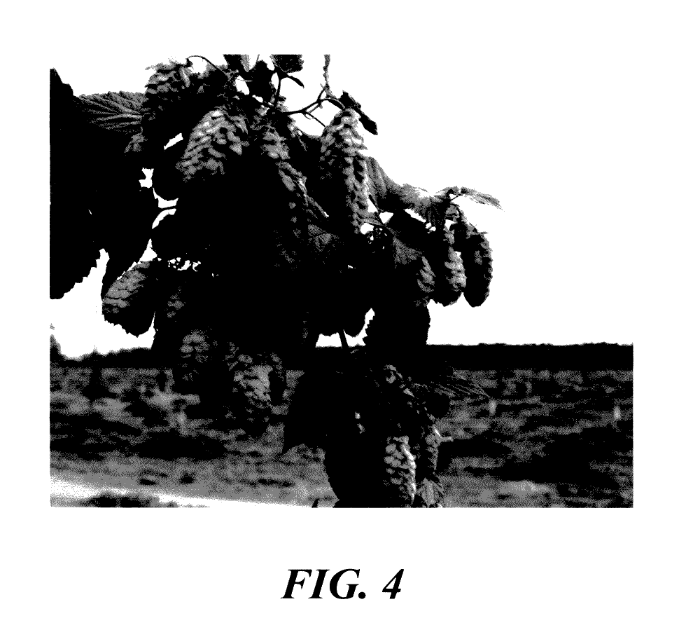

FIG. 5 illustrates leaves on a mature ‘HBC 522’ hop plant.

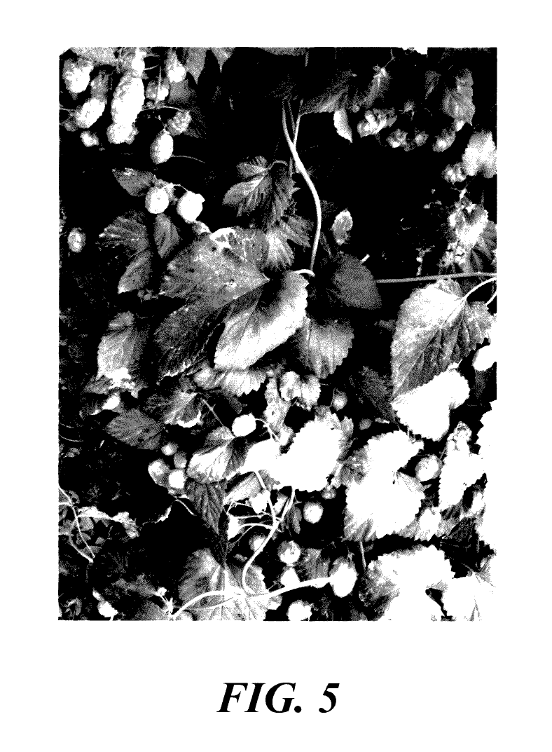

FIG. 6 illustrates leaves of the ‘HBC 522’ hop plant.

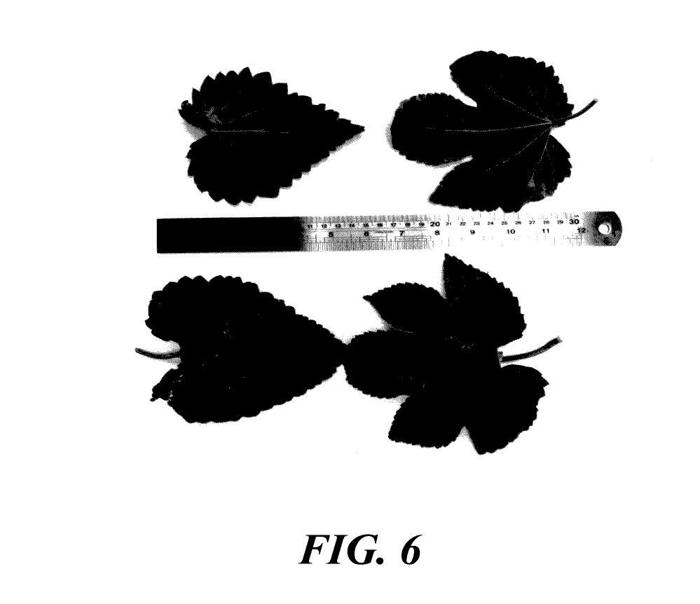

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2009-2015 growing seasons at Toppenish, Washington. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. All color references are based on The Royal Horticultural Society Color Chart.
Ploidy: Diploid (2n=2x).
Sex: Female.
Use: Brewing beer and ale.
Harvest date: September (during 2009 to 2015 growing seasons at Toppenish, Wash.)
Oil analytical characteristics (Averages):
   Total Oil (ml/100 g of cones).—230% Oil.
   A-Pinene.—0.04% Total Oil.
   B-Pinene.—0.90% Total Oil.
   Myrcene.—36.95% Total Oil.
   2-methyl-butyl isobutyrate.—0.09% Total Oil.
   Limonene.—0.12% Total Oil.
   Linalool.—0.64% Total Oil.
   Caryophyllene.—8.44% Total Oil.
   Farnesene.—0.52% Total Oil.
   Humulene.—13.18% Total Oil.
   Citral.—1.12% Total Oil.
   Geranyl acetate.—0.16% Total Oil.
   Citronellol.—3.36% Total Oil.
   Nerol.—0.39% Total Oil.
   Geraniol.—0.05% Total Oil.
   B-lonene.—0.03% Total Oil.
Bine:
   Color.—Green 143B.
   Stripe present.—Yes.
   Stripe color.—Red 42B.
   Stipule direction.—Horizontal.
   Bine diameter.—11 mm at base; 10 mm at nine feet; 9 mm at terminal end of eighteen feet.
Leaf:
   Arrangement.—Opposite.
   Shape.—Palmate.
   Average length of mature leaf.—13 cm.
   Average width of mature leaf.—11.5 cm.
   Color of mature leaf upper surface.—Green 136A.
   Color of mature leaf lower surface.—Green 137B.
   Color of immature leaf upper surface.—Green 131A.
   Color of immature leaf lower surface.—Green 132B.
   Number of lobes.—1-3.
   Margin.—Serrate.
   Serrations per inch.—3.
   Average petiole length (mature).—50 mm.
   Petiole color at base.—Yellow-Green 144B.
   Venation.—Palmate.
   Vein color.—Green 138A.
Cone:
   Avg. length.—34 mm.
   Avg. diameter.—20 mm.
   Bract tip color.—Green 141A.
   Bract base color.—Green 142A.
   Bracteole color.—Yellow-Green 150C.
   Cone shape.—Oblong.
   Bract shape.—Orbicular.
   Bract tip shape.—Cuspidate.
   Bracteole shape.—Ovate.

What is claimed is:

1. A new and distinct hop plant as shown and described herein.
